(19) United States
(12) Patent Application Publication
(10) Pub. No.: US 2026/0076926 A1
(43) Pub. Date: Mar. 19, 2026

(54) COMPOUNDS FOR USE IN ORGAN PRESERVATION AND ORGAN TRANSPLANTATION

(71) Applicant: ABREXA PHARMACEUTICALS, INC., San Diego, CA (US)

(72) Inventors: Jesus Ernesto VILLAFRANCA, San Diego, CA (US); Charles Richard KISSINGER, San Diego, CA (US)

(73) Assignee: ABREXA PHARMACEUTICALS, INC., San Diego, CA (US)

(21) Appl. No.: 19/397,818

(22) Filed: Nov. 21, 2025

Related U.S. Application Data
(63) Continuation of application No. PCT/US2024/031259, filed on May 28, 2024.
(60) Provisional application No. 63/469,205, filed on May 26, 2023.

(51) Int. Cl. A61K 31/167; A01N 1/126

(52) U.S. Cl. CPC A61K 31/167; A01N 1/126

(57) ABSTRACT

Presented herein, in certain aspects, are methods of preventing, reducing, delaying or inhibiting rejection, injury or partial or complete death of an organ prior to, during or after transplantation into an organ transplant recipient. The methods may comprise administering one or more compositions or compounds disclosed herein to an organ donor or to the organ transplant recipient. The methods may also comprise contacting an organ prior to, during and/or after transplantation, with one or more compositions or compounds disclosed herein.

### FIELD FOR USE IN A METHOD DESCRIBED HEREIN

The compositions, compounds and methods disclosed herein relate to the general field of organ transplantation, organ protection, organ preparation and organ preservation.

### BACKGROUND

Organ transplantation, in which a damaged or diseased organ is replaced with a relatively healthy organ from a donor, is the only available treatment for end-stage organ failure. The most commonly transplanted organ is the kidney, followed by liver, heart, and lung. Pancreas, intestine, and multi-organ transplants are also performed, though in much smaller numbers. Overall, more than 36,000 organ transplants were performed in the United States alone in 2018. Due to a shortage of suitable donor organs, however, the number of persons in the US in need of a transplanted organ currently exceeds 113,000 as of April 2019 (Data from United Network for Organ Sharing).

Beyond the shortage of donor organs, the central challenge in organ transplantation is rejection of the transplanted organ by the immune system of the recipient. Depending on the organ, between about one-quarter and one-half of transplants fail within five years (data from the United Network for Organ Sharing, Apr. 17, 2019). In an effort to prevent potential organ rejection, organ recipients often undergo long-term treatment with immunosuppressive drugs, which further endanger the health of the patient by increasing their susceptibility to infectious diseases and certain cancers. Such immunosuppressive treatments are also associated with other potentially serious side effects.

Transplant rejection has been classified into three major types with different time-scales and underlying mechanisms. The three major types are hyperacute rejection, acute rejection and chronic rejection. Hyperacute rejection occurs when there are pre-existing antibodies to the transplanted organ and can manifest itself within minutes after transplant. Hyperacute rejection is rarely encountered, however, because of routine pre-screening of recipients for anti-donor antibodies.

Acute rejection occurs within the first several months after transplant. Acute rejection occurs when the immune system of the recipient reacts to antigens, primarily those of the major histocompatibility complex (MHC), on the surfaces of the donor organ cells. The resulting inflammatory processes eventually result in necrosis of the graft tissue.

Chronic rejection occurs months or years after transplant. Chronic rejection is characterized by multiple vascular pathologies, including arteriosclerosis and progressive narrowing of graft vessels (graft vascular disease) often accompanied by fibrosis within the parenchyma of the transplanted organ. The resulting ischemia often causes necrosis of the transplanted tissue and eventual organ failure. Treatment of organ recipients with immunosuppressive drugs appears to have little effect on chronic rejection.

In addition to the closeness of the antigenic match between donor and recipient, the condition of a donor organ at the time of transplant is believed to have a major influence on both acute and chronic rejection. Organs from live donors generally have better survival rates (United Network for Organ Sharing). However, currently more than 80% of all organs are from deceased donors (United Network for Organ Sharing). Processes that occur both after the death of the donor and during the removal and handling of the organ prior to transplant can potentially affect the recipient immune response.

Organ donors who have undergone brain death are generally preferred over those who succumbed to cardiac death because the organ is infused with blood up until the moment of removal. However, brain death has been shown to result in significant, prolonged systemic inflammatory responses that can damage potential donor organs. Recent evidence indicates that these inflammatory processes have a negative effect on transplant outcomes, and that targeting the inflammatory factors produced after brain death in donors prior to transplantation is a potential strategy for improving transplant success rates.

Organs harvested for transplant are also subject to damage through ischemia-reperfusion injury. After removal from the donor, organs are typically maintained in cold storage in a preservative solution. Ischemia-reperfusion injury occurs when blood supply to the organ is removed and then returns after the organ is placed in the transplant recipient. Ischemia-reperfusion injury appears to contribute both to acute and chronic rejection in kidney transplantation.

### SUMMARY

In certain aspects, provided herein is a method of preventing, reducing, delaying or inhibiting transplant rejection in a subject comprising administering (i) to the subject (e.g., an organ transplant recipient), (ii) to an organ, and/or (iii) to an organ donor, a composition comprising a compound disclosed herein. In some embodiments, a compound for use in a method described herein comprises the structure of Formula IV;

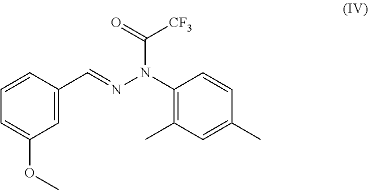 [SMILES: COc1cccc(/C=N/N(C(=O)C(F)(F)F)c2ccc(C)cc2C)c1]

or a pharmaceutically acceptable salt, stereoisomer or tautomer thereof.

### DETAILED DESCRIPTION FOR USE IN A METHOD DESCRIBED HEREIN

Presented herein are, in certain aspects, are methods of preventing, reducing, delaying or inhibiting rejection, injury or partial or complete death of an organ prior to, during or after transplantation of the organ into a transplant recipient. The methods may comprise administering one or more compositions or compounds disclosed herein to an organ donor or to an organ transplant recipient. The methods may also comprise contacting an organ prior to, during and/or after transplantation, with one or more compositions or compounds disclosed herein.

### Compounds

In some embodiments, a compound for use in a method described herein comprises the structure of Formula I;

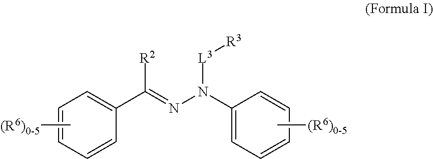

or a pharmaceutically acceptable salt, stereoisomer or tautomer thereof. In some embodiments of Formula I, R² is hydrogen (H) or methyl; R³ is a methyl, a fluorine substituted alkyl (e.g., fluoromethyl, difluoromethyl, or trifluoromethyl), or a bromine substituted alkyl (e.g., bromomethyl, dibromomethyl, tribromomethyl); L³ is a carbonyl; and R⁶ at each occurrence is independently selected from alkyl, substituted alkyl, alkenyl, substituted alkenyl, cycloalkyl, substituted cycloalkyl, hydroxyl, methoxy, alkoxy, substituted alkoxy, aryloxy, substituted aryloxy, mercapto, alkylthio, arylthio, carbonyl, carboxyl, aryl, substituted aryl, substituted heterocyclic, halogen, cyano, cyanoalkyl, amine, methyl amine, dimethyl amine, nitro, amino, amidino, carbamate, CF₃, OCF₃, S(O)ₙR⁷, and C(O)R⁸, or two R⁶ at adjacent positions combine to form an optionally substituted heteroaryl or heteroalkyl ring fused with the adjoining phenyl moiety; where R⁷ is selected from H, R⁹, NH₂, HNR⁹ and NR⁹R¹⁰; R⁸ is selected from OH, OR⁹, NH₂, NHR⁹ and NR⁹R¹⁰, where R⁹ and R¹⁰ at each occurrence are independently an optionally substituted alkyl; and n is 1 or 2.

In certain embodiments of Formula I, R⁶ at each occurrence is independently selected from alkyl, substituted alkyl, alkenyl, substituted alkenyl, hydroxyl, alkoxy, methoxy, substituted alkoxy, halogen, carbonyl, carboxyl, or C(O)R⁸; and in certain such aspects, R⁶ at each occurrence is methyl, methoxy, perfluoromethyl, perfluoromethoxy, hydroxyl, Cl, F, or I. In some embodiments of Formula I, L³ is carbonyl, R³ is CF₃, R² is H, and R⁶ is null or H at every occurrence. In some embodiments of Formula I, L³ is carbonyl, R³ is CF₃, R² is H, and R⁶ is independently selected from methyl or methoxy, at each occurrence. In some embodiments of Formula I, L³ is carbonyl, R³ is CF₃, R² is methyl, and R⁶ is independently selected from methyl or methoxy, at each occurrence.

In some embodiments, a compound for use in a method described herein comprises the structure of Formula II;

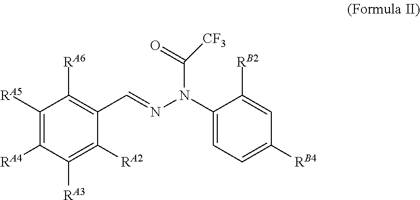

or a pharmaceutically acceptable salt, stereoisomer or tautomer thereof, where:
(i) R^{A2}, R^{A4}, R^{A5}, and R^{A6} is H, R^{A3} is methoxy, R^{B2} is methyl, and R^{B4} is methyl;
(ii) R^{A2}, R^{A3}, R^{A5}, and R^{A6} is H, R^{A4} is methoxy, R^{B2} is methyl, and R^{B4} is methyl;
(iii) R^{A2}, R^{A3}, R^{A4}, R^{A5}, and R^{A6} is H, R^{B2} is H, and R^{B4} is H;
(iv) R^{A2}, R^{A3}, R^{A4}, R^{A5}, and R^{A6} is H, R^{B2} is methyl, and R^{B4} is methyl;
(v) R^{A2}, R^{A4}, R^{A5}, and R^{A6} is H, R^{A3} is methoxy, R^{B2} is H, and R^{B4} is H;
(vi) R^{A2}, R^{A3}, R^{A4}, R^{A5}, and R^{A6} is H, R^{B2} is H, and R^{B4} is methyl;
(vii) R^{A2}, R^{A4}, R^{A5}, and R^{A6} is H, R^{A3} is methoxy, R^{B2} is H, and R^{B4} is methyl;
(viii) R^{A2}, R^{A3}, R^{A4}, R^{A5}, and R^{A6} is H, R^{B2} is methyl, and R^{B4} is H;
(ix) R^{A2}, R^{A4}, R^{A5}, and R^{A6} is H, R^{A3} is methoxy, R^{B2} is methyl, and R^{B4} is H;
(x) R^{A2}, R^{A3}, R^{A5}, and R^{A6} is H, R^{A4} is COOH, R^{B2} is methyl, and R^{B4} is methyl;
(xi) R^{A2}, R^{A4}, and R^{A5} is H, R^{A3} and R^{A6} is hydroxyl, R^{B2} is methyl, and R^{B4} is methyl;
(xii) R^{A2}, R^{A4}, and R^{A6} is H, R^{A3} and R^{A5} is hydroxyl, R^{B2} is methyl, and R^{B4} is methyl;
(xiii) R^{A2}, R⁴⁴, and R^{A5} is H, R^{A3} is methoxy, R^{A6} is F, R^{B2} is H, and R^{B4} is Cl;
(xiv) R^{A3} and R^{A5} is H, R^{A2} and R^{A6} is F, R^{A4} is hydroxyl, R^{A6} is F, R^{B2} is H, and R^{B4} is F;
(XV) R^{A2}, R^{A4}, and R^{A6} is H, R^{A3} is hydroxyl, R^{A5} is F, R^{B2} is H, and R^{B4} is F; or
(xvi) R^{A2}, R^{A5}, and R^{A6} is H, R^{A3} and R⁴⁴ taken together are —O—CH₂—O—, R^{A5} is F, R^{B2} is H, and R^{B4} is F.

In some embodiments of the compound of Formula II, R^{A2}, R^{A5}, and R^{A6} are H, R^{A3} is methoxy, R^{B2} and R^{B4} are methyl, and R^{A4} is selected from H, NO₂, OH, methoxy, phenol, methyl, Fluorine (F), N(CH₃)₂, CHC(CN)₂ and O-tert-butyldimethylsilyl (OTBDMS). In some embodiments of the compound of Formula II, R^{A2}, R^{A4}, R^{A5}, and R^{A6} are H, R^{A3} is methoxy, R^{B2} is methyl, and R^{B4} is methyl. In some embodiments of the compound of Formula II, R^{A2}, R^{A3}, R^{A5}, and R^{A6} are H, R^{A4} is methoxy, R^{B2} is methyl, and R^{B4} is methyl. In some embodiments of the compound of Formula II, R^{A2}, R^{A3}, R^{A4}, R^{A5} and R^{A6} are H, R^{B2} is methyl, and R^{B4} is methyl. In some embodiments of the compound of Formula II, R^{A2}, R^{A4}, R^{A5} and R^{A6} are H, R^{A3} is methoxy, R^{B2} is H, and R^{B4} is H. In some embodiments of the compound of Formula II, R^{A2}, R^{A3}, R^{A4}, R^{A5} and R^{A6} are H, R^{B2} is H, and R^{B4} is methyl. In some embodiments of the compound of Formula II, R^{A2}, R^{A3}, R^{A4}, R^{A5} and R^{A6} are H, R^{B2} is H, and R^{B4} is methyl. In some embodiments of the compound of Formula II, R^{A2}, R^{A4}, RAS and R^{A6} are H, R^{A3} is methoxy, R^{B2} is H, and R^{B4} is methyl. In some embodiments of the compound of Formula II, R^{A2}, R^{A4}, R^{A5} and R^{A6} are H, R^{A3} is methoxy, R^{B2} is methyl, and R^{B4} is H. In some embodiments of the compound of Formula II, R^{A2}, R^{A3}, R^{A4}, R^{A5} and R^{A6} are H, R^{B2} is methyl, and R^{B4} is H. In some embodiments of the compound of Formula II, R^{A2}, R^{A3}, R^{A5} and R^{A6} are H, R^{A4} is a carboxyl, R^{B2} is methyl, and R^{B4} is methyl. In some embodiments of the compound of Formula II, R^{A2}, R^{A4}, R^{A5} and R^{A6} are H, R^{A3} is a carboxyl, R^{B2} is methyl, and R^{B4} is methyl.

In some embodiments, a compound for use in a method described herein comprises the structure of Formula VI;

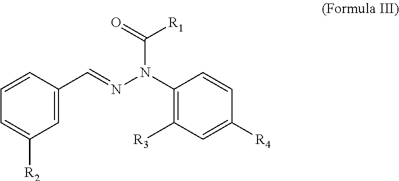

or a pharmaceutically acceptable salt, stereoisomer or tautomer thereof, where R₁ is methyl, fluoromethyl, difluoromethyl, trifluoromethyl, bromomethyl, dibromomethyl or tribromomethyl; R² is methyl, methoxy, hydroxyl, halogen, CF₃, OCH₃, OCF₃ or OCBr₃; and R³ and R⁴ are independently selected from hydrogen, hydroxyl, a halogen (e.g., Cl, F or Br), methyl, a methoxy, and an amine. In some embodiments of Formula III, R¹ is CF₃ (trifluoromethyl), R² is OCH₃, and R³ and R⁴ are methyl. In some embodiments of Formula III, R¹ is CF₃ (trifluoromethyl), R² is OCF₃, and R³ and R⁴ are methyl.

In some embodiments, a compound for use in a method described herein comprises the structure of Formula IV below, or a pharmaceutically acceptable salt, stereoisomer or tautomer thereof.

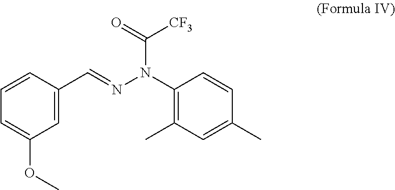 [SMILES: COc1cccc(/C=N/N(C(=O)C(F)(F)F)c2ccc(C)cc2C)c1]

The following terms have the respective definitions set out below.

“Alkyl” refers to straight or branched chain alkyl radicals having in the range of about 1 up to about 12 carbon atoms (e.g., methyl, ethyl, propyl, butyl, and the like). “Substituted alkyl” refers to alkyl further bearing one or more substituents (e.g., 1, 2, 3, 4, or even 5) as set forth herein. “Optionally substituted alkyl” refers to alkyl or substituted alkyl.

“Cycloalkyl” refers to cyclic ring-containing groups containing in the range of about 3 up to about 12 carbon atoms. “Substituted cycloalkyl” refers to cycloalkyl further bearing one or more substituents (e.g., 1, 2, 3, 4, or even 5) selected from alkyl, substituted alkyl, as well as any of the substituents set forth herein. “Optionally substituted cycloalkyl” refers to cycloalkyl or substituted cycloalkyl.

“Heterocycle,” “heterocyclic” and like terms refer to cyclic (i.e., ring-containing) groups containing one or more heteroatoms (e.g., N, O, S, or the like) as part of the ring, and having in the range of 1 up to about 14 carbon atoms. “Substituted heterocyclic” and like terms refer to heterocycle further bearing one or more substituents (e.g., 1, 2, 3, 4, or even 5) as set forth herein. Exemplary heterocyclic moieties include saturated rings, unsaturated rings, and aromatic heteroatom-containing ring systems, e.g., epoxy, tetrahydrofuran, oxazoline, pyrrole, pyridine, furan, and the like. “Optionally substituted heterocycle” and like terms refer to heterocycle or substituted heterocycle.

Reference to “optionally substituted bicyclic ring” refers to a bicyclic ring structure as known in the art, optionally including substitutions as defined herein.

“Alkenyl” refers to straight, branched chain, or cyclic hydrocarbyl groups including from 2 to about 20 carbon atoms having at least one, 1-3, 1-2, or one, carbon to carbon double bond. “Substituted alkenyl” refers to alkenyl substituted at 1 or more, e.g., 1, 2, 3, 4, or even 5 positions, with substitution as described herein. “Optionally substituted alkenyl” refers to alkenyl or substituted alkenyl. In some embodiments, an alkenyl is ethylenyl or propylenyl. In certain embodiments, a substituted alkenyl is a substituted ethylenyl or substituted propylenyl. In some embodiments, ethylenyl or propylenyl is substituted with one or more CN moieties. For example, in some embodiments, a substituted ethylenyl comprises (CN)₂C═CH—.

“Aryl” refers to aromatic groups having in the range of 6 up to about 14 carbon atoms. “Substituted aryl” refers to aryl radicals further bearing one or more substituents (e.g., 1, 2, 3, 4, or even 5) selected from alkyl, substituted alkyl, cycloalkyl, substituted cycloalkyl, hydroxyl, alkoxy, aryloxy, mercapto, alkylthio, arylthio, carbonyl, aryl, substituted aryl, heterocyclic, substituted heterocyclic, halogen, trifluoromethyl, pentafluoroethyl, cyano, cyanoalkyl, nitro, amino, amido, amidino, carboxyl, carbamate, SO₂X, wherein X is H, R, NH₂, NHR or NR₂, SO₃Y, wherein Y is H, NH₂, NHR or NR₂, or C(O)Z, wherein Z is OH, OR, NH₂, NHR or NR₂, and the like. “Optionally substituted aryl” refers to aryl or substituted aryl.

“Aralkyl” refers to an alkyl group substituted by an aryl group. “Substituted aralkyl” refers to aralkyl further bearing one or more substituents (e.g., 1, 2, 3, 4, or even 5) selected from alkyl, substituted alkyl, cycloalkyl, substituted cycloalkyl, as well as any of the substituents set forth herein. Thus, aralkyl groups include benzyl, diphenylmethyl, and 1-phenylethyl (—CH(C₆H₅)(CH₃)) among others. “Optionally substituted aralkyl” refers to aralkyl or substituted aralkyl.

“Heteroaryl” refers to aromatic groups containing one or more heteroatoms (e.g., N, O, S, or the like) as part of the aromatic ring, typically having in the range of 2 up to about 14 carbon atoms, and “substituted heteroaryl” refers to heteroaryl radicals further bearing one or more substituents (e.g., 1, 2, 3, 4, or even 5) selected from alkyl, substituted alkyl, cycloalkyl, substituted cycloalkyl, as well as any of the substituents set forth above.

“Heteroaralkyl” and “heteroarylalkyl” refer to an alkyl group substituted by one or more heteroaryl groups. “Substituted heteroaralkyl” refers to heteroaralkyl further bearing one or more substituents (e.g., 1, 2, 3, 4, or even 5) selected from alkyl, substituted alkyl, cycloalkyl, substituted cycloalkyl, as well as any of the substituents set forth herein.

“Optionally substituted heteroaralkyl” refers to heteroaralkyl or substituted heteroaralkyl.

“Halogen” and “halo” refer to fluorine, chlorine, bromine or iodine.

“Hydroxyl” and “hydroxy” refer to the functionality —OH.

“Alkoxy” denotes the group —OR, where R is alkyl. “Substituted alkoxy” denotes the group —OR, where R is substituted alkyl. “Optionally substituted alkoxy” refers to alkoxy or substituted alkoxy.

“Aryloxy” denotes the group —OR, where R is aryl. “Substituted aryloxy” denotes the group —OR, where R is substituted aryl. “Optionally substituted aryloxy” refers to aryloxy or substituted aryloxy.

“Mercapto” and “thiol” refer to the functionality —SH.

“Alkylthio” and “thioalkoxy” refer to the group —SR, —S(O)ₙ₌₁₋₂—R, where R is alkyl. “Substituted alkylthio” and “substituted thioalkoxy” refers to the group —SR, —S(O)ₙ₌₁₋₂—R, where R is substituted alkyl. “Optionally substituted alkylthio” and “optionally substituted thioalkoxy” refers to alkylthio or substituted alkylthio.

“Arylthio” denotes the group —SR, where R is aryl. “Substituted arylthio” denotes the group —SR, where R is substituted aryl. “Optionally substituted arylthio” refers to arylthio or substituted arylthio.

“Amino” refers to unsubstituted, monosubstituted and disubstituted amino groups, including the substituent-NH₂, “monoalkylamino,” which refers to a substituent having structure —NHR, wherein R is alkyl or substituted alkyl, and “dialkylamino,” which refers to a substituent of the structure —NR₂, wherein each R is independently alkyl or substituted alkyl.

“Amidino” denotes the group —C(═NR^{q})NR^{r}R^{s}, wherein R^{q}, R^{r}, and R^{s} are independently hydrogen or optionally substituted alkyl.

Reference to “amide group” embraces substituents of the structure —C(O)—NR₂, wherein each R is independently H, alkyl, substituted alkyl, aryl or substituted aryl as set forth above. When each R is H, the substituent is also referred to as “carbamoyl” (i.e., a substituent having the structure —C(O)—NH₂). When only one of the R groups is H, the substituent is also referred to as “monoalkylcarbamoyl” (i.e., a substituent having the structure —C(O)—NHR, wherein R is alkyl or substituted alkyl as set forth above) or “arylcarbamoyl” (i.e., a substituent having the structure —C(O)—NH (aryl), wherein aryl is as defined above, including substituted aryl). When neither of the R groups are H, the substituent is also referred to as “di-alkylcarbamoyl” (i.e., a substituent having the structure —C(O)—NR₂, wherein each R is independently alkyl or substituted alkyl as set forth above).

Reference to “carbamate” embraces substituents of the structure —O—C(O)—NR₂, wherein each R is independently H, alkyl, substituted alkyl, aryl or substituted aryl.

Reference to “ester group” embraces substituents of the structure —O—C(O)—OR, wherein each R is independently alkyl, substituted alkyl, aryl or substituted aryl.

“Acyl” refers to groups having the structure —C(O)R, where R is hydrogen, alkyl, aryl, and the like as defined herein. “Substituted acyl” refers to acyl wherein the substituent R is substituted as defined herein. “Optionally substituted acyl” refers to acyl and substituted acyl.

“Cyanoalkyl” refers to the group —R≡N, wherein R is an optionally substituted alkylenyl.

As used here, “substitution” denotes an atom or group of atoms that has been replaced with another atom or group of atoms (i.e., substituent), and includes all levels of substitution, e.g., mono-, di-, tri-, tetra-, penta-, or even hex-substitution, where such substitution is chemically permissible. Substitutions can occur at any chemically accessible position and on any atom, such as substitution(s) on carbon and any heteroatom, such as oxygen, nitrogen, or sulfur. For example, substituted moieties include those where one or more bonds to a hydrogen or carbon atom(s) contained therein are replaced by a bond to non-hydrogen and/or non-carbon atom(s). Substitutions can include, but are not limited to, a halogen atom such as F, Cl, Br, and I; an oxygen atom in groups such as hydroxyl groups, alkoxy groups, aryloxy groups, and ester groups; a sulfur atom in groups such as thiol groups, alkyl and aryl sulfide groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and heteroatoms in other groups as well known in the art.

Non-limiting examples of substituents include, without limitation, halogen, —OH, —NH₂, —NO₂, —CN, —C(O)OH, —C(S)OH, —C(O)NH₂, —C(S)NH₂, —S(O)₂NH₂, —NHC(O)NH₂, —NHC(S)NH₂, —NHS(O)₂NH₂, —C(NH)NH₂, —OR, —SR, —OC(O)R, —OC(S)R, —C(O)R, —C(S)R, —C(O)OR, —C(S)OR, —S(O)R, —S(O)₂R, —C(O)NHR, —C(S)NHR, —C(O)NRR, —C(S)NRR, —S(O)₂NHR, —S(O)₂NRR, —C(NR) NHR, —C (NH)NRR, —NHC(O)R, —NHC(S)R, —NRC(O)R, —NRC(S)R, —NHS(O)₂R, —NRS(O)₂R, —NHC(O)NHR, —NHC(S)NHR, —NRC(O)NH₂, —NRC(S)NH₂, —NRC(O)NHR, —NRC(S)NHR, —NHC(O)NRR, —NHC(S)NRR, —NRC(O)NRR, —NRC(S)NRR, —NHS(O)₂NHR, —NRS(O)₂NH₂, —NRS(O)₂NHR, —NHS(O)₂NRR, —NRS(O)₂NRR, —NHR, —NRR, where R at each occurrence is independently H, optionally substituted alkyl, optionally substituted aryl, or optionally substituted heteroaryl. Also contemplated is substitution with an optionally substituted hydrocarbyl moiety containing one or more of the following chemical functionalities: —O—, —S—, —NR—, —O—C(O)—, —O—C(O)—O—, —O—C(O)—NR—, —NR—C(O)—, —NR—C(O)—O—, —NR—C(O)—NR—, —S—C(O)—, —S—C(O)—O—, —S—C(O)—NR—, —S(O)—, —S(O)₂—, —O—S(O)₂—, —O—S(O)₂—O, —O—S(O)₂—NR—, —O—S(O)—, —O—S(O)—O—, —O—S(O)—NR—, —O—NR—C(O)—, —O—NR—C(O)—O—, —O—NR—C(O)—NR—, —NR—O—C(O)—, —NR—O—C(O)—O—, —NR—O—C(O)—NR—, —O—NR—C(S)—, —O—NR—C(S)—O—, —O—NR—C(S)—NR—, —NR—O—C(S)—, —NR—O—C(S)—O—, —NR—O—C(S)—NR—, —O—C(S)—, —O—C(S)—O—, —O—C(S)—NR—, —NR—C(S)—, —NR—C(S)—O—, —NR—C(S)—NR—, —S—S(O)₂—, —S—S(O)₂—O—, —S—S(O)₂—NR—, —NR—O—S(O)—, —NR—O—S(O)—O—, —NR—O—S(O)—NR—, —NR—O—S(O)₂—, —NR—O—S(O)₂—NR—, —O—NR—S(O)—, —O—NR—S(O)—O—, —O—NR—S(O)—NR—, —O—NR—S(O)₂—O—, —O—NR—S(O)₂—NR—, —O—NR—S(O)₂—, —O—P(O)R²—, —S—P(O)R²—, or —NRP(O)R²—, where R at each occurrence is independently H, optionally substituted alkyl, optionally substituted aryl, or optionally substituted heteroaryl.

In some embodiments, a compound for use in a method described herein includes isomers including stereoisomers (e.g., enantiomer and diasteromers), constitutional isomers, tautomers, conformational isomers, and geometric isomers of a compound disclosed herein.

Exemplary constitutional isomers include for example without limitation, isomers resulting from different connectivity of functionalities forming the compounds for use in a method described herein, for example, 1-propyl versus 2-propyl substitution, and the like. Constitutional isomers in combination with tautomerization additionally embrace bonding rearrangements involving the migration of double bonds and substituents. For example, tautomerization in combination with a 1-3 pleiotropic hydrogen shift can result in constitutional isomerism.

Exemplary conformational isomers include for example without limitation, isomers produced by rotation about a bond wherein the rotation is hindered to the extent that separable isomers result, as well known in the art.

Exemplary geometrical isomers include double bonds in e.g., the “E” or “Z” configuration, as well known in the art.

Compounds for use in a method described herein can be readily prepared using a suitable synthetic method. For example, curcumin can be condensed with phenyl hydrazine by warming to reflux overnight in toluene. Optionally, a catalytic amount of acid (HCl) can be employed. In some embodiments, pure curcumin (vs. technical grade) and freshly distilled phenyl hydrazine can be employed.

As another example, 3-methoxy benzaldehyde can be condensed with 2,4-dimethylphenyl hydrazine in methanol employing standard hydrazone preparation conditions (e.g., heating in the microwave to speed the reaction time). Next, the free NH is acylated with TFAA (trifluoroacetic anhydride) plus catalytic (0.1%) amounts of DMAP (dimethylamino pyridine), THF (tetrahydrofuran) or DCM (dichloromethane).

In some embodiments, CF₃ substituted triazoles can be prepared by 1,3-dipolar cycloaddition between suitable aryltrifluoromethylacetylenes and aryl azides. Regioselectivity can be obtained by utilizing a suitable click chemistry (e.g., see Huisgen R. (1984) 1,3-Dipolar Cycloaddition Chemistry, pp. 1-176, Lodon: Wiley; Padwa (1991) Comprehensive Organic Synthesis, Vol. 4: pp. 1069-1109, Oxford: Pergamon; and Fan & Katritzky (1996) Comprehensive Heterocyclic Chemistry II, Vol. 4: pp. 101-126, Oxford: Pergamon). Additional methods of generating compounds disclosed herein can be found in Lima et al., (2015) Chem. Commun. 51:10784-10796 and Kim et al., (2015) Org. Biomol. Chem. 13:9564-9569.

In some embodiments, a compound for use in a method described herein is provided in the form of pharmaceutically acceptable salt. A compound for use in a method described herein can be complexed with any suitable inorganic or organic salt. In some embodiments, a salt of a compound for use in a method described herein is prepared by reacting a compound for use in a method described herein with a suitable organic or inorganic acid or base. Non-limiting examples of organic salts contemplated for use in a method described herein with a compound for use in a method described herein include methanesulfonate, acetate, oxalate, adipate, alginate, aspartate, valerate, oleate, laurate, borate, benzoate, lactate, phosphate, toluenesulfonate (tosylate), citrate, malate, maleate, fumarate, succinate, tartrate, napsylate, methanesulfonate, 2-naphthalenesulfonate, nicotinate, benzenesulfonate, butyrate, camphorate, camphorsulfonate, cyclopentanepropionate, digluconate, dodecylsulfate, glucoheptanoate, glycerophosphate, heptanoate, hexanoate, undecanoate, 2-hydroxyethanesulfonate, ethane sulfonate, and the like. In some embodiments, inorganic salts can be formed from inorganic acids such as sulfate, bisulfate, hemisulfate, hydrochloride, chlorate, perchlorate, hydrobromide, hydroiodide, and the like. Non-limiting examples of a base salt include ammonium salts; alkali metal salts such as sodium salts, potassium salts, and the like; alkaline earth metal salts such as calcium salts, magnesium salts, and the like; salts with organic bases such as dicyclohexylamine salts, N-methyl-D-glucamine, phenylethylamine, and the like; and salts with amino acids such as arginine, lysine, and the like. Salt forms of a compound for use in a method described herein can be prepared employing a suitable method.

Presented herein, in some embodiments, is a method for inhibiting, reducing, delaying or preventing rejection of a transplanted organ in a subject comprising administering to the subject a composition comprising a therapeutically effective amount of one or more compounds disclosed herein. In some embodiments, the one or more compounds are selected from Formula I, II, III and IV, or a pharmaceutically acceptable salt, stereoisomer or tautomer thereof. In some embodiments, the subject is a recipient of an organ transplant. In some embodiments, the subject is in need of an organ transplant or is about to undergo an organ transplantation. The composition, in certain embodiments, is administered to the subject prior to, during and/or after an organ transplant procedure. For example, a composition disclosed herein may be administered to a subject within seconds to weeks prior to an organ transplant procedure. In some embodiments, a composition is administered to a subject 1 to 10 weeks, 1 to 30 days, 1 to 14 days, or 1 to 3 days prior to an organ transplant procedure. In some embodiments, a composition is administered to a subject 1 to 48 hours prior to an organ transplant procedure. In some embodiments, a composition is administered to a subject at least 48 hours, at least 24 hours, at least 12 hours, least 1 hour or at least 1 minute prior to an organ transplant procedure. In some embodiments, a composition is administered to a subject during a transplant procedure. In some embodiments, a composition is administered to a subject for at least 1 to 48 hours, at least 1 to 30 days, for at least 1 to 10 weeks or for several months or years following an organ transplant procedure.

In some embodiments, a method for inhibiting, reducing, delaying or preventing rejection of a transplanted organ in a subject comprising inhibiting, reducing, delaying or preventing acute rejection of a transplanted organ. In some embodiments, a method for inhibiting, reducing, delaying or preventing rejection of a transplanted organ in a subject comprising inhibiting, reducing, delaying or preventing chronic rejection of a transplanted organ.

In some embodiments presented herein is an ex vivo or in vitro method of preparing an organ for transplantation into a subject comprising contacting the organ with a composition comprising an effective amount of a compound selected from any one of Formulas I to IV, or a pharmaceutically acceptable salt, stereoisomer or tautomer thereof. In some or all of the embodiments of the ex vivo methods described herein, the organ is contacted with a composition or compound after the organ is removed from a subject (e.g., from an organ donor) and/or prior to transplantation into an organ transplant recipient. In some or all of the embodiments of an ex vivo method described herein, an organ that is contacted with a composition is an organ that is not connected or attached to a subject (e.g., a human body). In some or all of the embodiments of an ex vivo method described herein, an organ that is contacted with a composition is an organ that is not operably connected to a subject (e.g., a human body).

In some embodiments, a process of contacting an organ with a composition described herein comprises perfusion of the organ with the composition. In some embodiments, a process of contacting an organ with a composition described herein comprises partially or completely submerging the organ in the composition. In some embodiments, a process of contacting an organ with a composition described herein comprises perfusion of the organ with the composition and partially or completely submerging the organ in the composition.

In some embodiments presented herein is an in situ method of preparing an organ for transplantation comprising contacting an organ with a composition comprising an effective amount of a compound selected from any one of Formulas I to IV, or a pharmaceutically acceptable salt, stereoisomer or tautomer thereof.

In some embodiments of an in situ method described herein, the subject is an organ donor. An organ donor may be alive, brain dead or deceased. In some embodiments of an in situ method described herein, the organ is contacted with a composition described herein prior to removal of the organ from the subject (e.g., from the donor). In some embodiments of an in situ method described herein, the organ is contacted with a composition described herein while the organ is operably connected to the subject. In some embodiments of an in situ method described herein, the organ is contacted with a composition described herein while the organ is operably connected to the subject in its naturally occurring anatomical configuration. In some embodiments of an in situ method described herein, the subject (e.g., donor) is alive. In some embodiments of an in situ method described herein, the subject (e.g., donor) is brain dead. In some embodiments of an in situ method described herein, the subject is deceased or has recently died (e.g., within minutes or within hours).

In some embodiments, a process of contacting an organ with a composition described herein comprises perfusion of the organ with the composition. In some embodiments, a process of contacting an organ with a composition described herein comprises partially or completely submerging the organ in the composition. In some embodiments, a process of contacting an organ with a composition described herein comprises perfusion of the organ with the composition and partially or completely submerging the organ in the composition. An organ located in or outside of a subject can be perfused with a composition described herein using a suitable method.

In some embodiments, presented herein is a method of inhibiting, reducing, delaying or preventing reperfusion injury of an organ transplanted in a subject (e.g., an organ transplant recipient) comprising administering to the subject a composition comprising a therapeutically effective amount of a compound comprising a structure selected from any one of Formulas I to IV, and/or contacting the organ with a composition comprising an effective amount of a compound comprising a structure selected from any one of Formulas I to IV. In some embodiments, presented herein is a method of inhibiting, reducing, delaying or preventing cell death of cells in an organ in a subject comprising administering to the subject a composition comprising a therapeutically effective amount of a compound comprising a structure selected from any one of Formulas I to IV and/or contacting the organ with a composition comprising an effective amount of a compound comprising a structure selected from any one of Formulas I to IV. The composition may be administered to the subject during, prior to, and/or after transplant of the organ into the subject. In some embodiments the organ is contacted with a composition disclosed herein by an ex vivo or in vitro method described herein. The organ may be contacted with the composition before, during or, in certain embodiments, after transplantation into a subject.

The term “cell death” includes cell death characterized by apoptosis, cell death characterized by necrosis, cell death characterized by necroptosis and/or cell death characterized by autophagy. In some embodiments, administering a composition disclosed herein to a subject inhibits, reduces, delays or prevents apoptosis of cells in an organ (e.g., a transplanted organ, or an organ intended for transplant). In some embodiments, administering a composition disclosed herein to a subject inhibits, reduces, delays or prevents necrosis of cells in an organ (e.g., a transplanted organ, or an organ intended for transplant). In some embodiments, administering a composition disclosed herein to a subject inhibits, reduces, delays or prevents necroptosis of cells in an organ (e.g., a transplanted organ, or an organ intended for transplant). In some embodiments, administering a composition disclosed herein to a subject inhibits, reduces, delays or prevents autophagy of cells in an organ (e.g., a transplanted organ, or an organ intended for transplant).

In some embodiments, presented herein is an ex vivo or in vitro method of inhibiting, reducing, delaying or preventing cell death of cells in an organ comprising contacting the organ with a composition comprising an effective amount of a compound comprising a structure selected from any one of Formulas I to IV. In some embodiments, presented herein is an in situ method of inhibiting, reducing, delaying or preventing cell death of cells in an organ comprising contacting the organ (e.g., an organ of a donor; e.g., a subject or cadaver) with a composition comprising an effective amount of a compound comprising a structure selected from any one of Formulas I to IV. In some embodiments, contacting an organ with a composition disclosed herein inhibits, reduces, delays or prevents apoptosis of cells in the organ (e.g., a transplanted organ, or an organ intended for transplant). In some embodiments, contacting an organ with a composition disclosed herein inhibits, reduces, delays or prevents necrosis of cells in the organ (e.g., a transplanted organ, or an organ intended for transplant). In some embodiments, contacting an organ with a composition disclosed herein inhibits, reduces, delays or prevents necroptosis of cells in the organ (e.g., a transplanted organ, or an organ intended for transplant). In some embodiments, contacting an organ with a composition disclosed herein inhibits, reduces, delays or prevents autophagy of cells in an organ (e.g., a transplanted organ, or an organ intended for transplant).

### Organs

An “organ” is any organ or part of a subject that is suitable for transplant into the same or a different subject, non-limiting examples of which include solid organs, tissues, body parts, cells and platelets. Non-limiting examples of solid organs include kidney, liver, heart, lung, pancreas, intestine, thymus, an eye, portions thereof, combinations thereof, and the like. Non-limiting examples of tissues include skin, bone, tendon, middle ear, cornea, heart valves, veins, cartilage, ligaments, portions thereof, combinations thereof, and the like. Non-limiting examples of body parts include a hand, a thumb, a finger, a toe, a foot, a face, a nose, an ear, an eye, an arm, a leg, portions thereof, combinations thereof, and the like. Non-limiting examples of cells include red blood cells, stem cells (e.g., cord blood stem cells, peripheral blood stem cells), bone marrow, portions thereof, combinations thereof, and the like.

### Subjects

The term “subject” refers to a mammal. Any suitable mammal can be treated by a method or composition described herein. Non-limiting examples of mammals include a human, non-human primate (e.g., ape, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a subject is a non-human primate or a human. In some embodiments a subject is a human. A subject can be any age or at any stage of development (e.g., an adult, teen, child, infant, or a mammal in utero). A subject can be male or female.

In some embodiments, a subject is an organ donor. In some embodiments an organ donor is alive. In some embodiments, an organ donor is brain dead. In some embodiments an organ donor is alive and brain dead. In some embodiments an organ donor is recently deceased. For example, in some embodiments, an organ donor that is recently deceased has been determined to be clinically dead for a period of seconds to hours (e.g., for up to 1 hour, up to 4 hours, up to 12 hours, or up to 24 hours). In some embodiments an organ donor is a cadaver.

In some embodiments, a subject is in need of an organ transplant. In some embodiments, a subject is a recipient of an organ transplant (e.g., an organ transplant recipient). In some embodiments, an organ transplant recipient is a subject who is about to have or is scheduled to have an organ transplant. In some embodiments, an organ transplant recipient is a subject who is having and/or has had an organ transplant. In some embodiments, an organ transplant recipient is alive. An organ transplant recipient is not a subject that is deceased, is not a subject that is dead, and/or is not a cadaver.

### Compositions

In some embodiments, a composition or pharmaceutical composition comprises one or more compounds described herein. In some embodiments, a composition or pharmaceutical composition consists essentially of one or more compounds described herein. For example, a composition consisting essentially of a compound described herein does not include any other active pharmaceutical ingredients (API), but may include excipients, diluents, additives or carriers. In some embodiments, a composition or pharmaceutical composition comprises an effective amount or a therapeutically effective amount of a compound described herein. In some embodiments provided herein is a pharmaceutical composition comprising a compound described herein for use in inhibiting, reducing, delaying or preventing rejection of a transplanted organ; for use in inhibiting, reducing, delaying or preventing reperfusion injury of a transplanted organ; and/or for inhibiting, reducing, delaying or preventing cell death of cells in a transplanted organ. In some embodiments, a pharmaceutical composition comprises a compound described herein and a pharmaceutically acceptable excipient, diluent, additive or carrier.

In some embodiments, a pharmaceutical composition is formulated for a suitable route of administration. In some embodiments, a pharmaceutical composition is formulated for contacting an organ. In some embodiments a pharmaceutical composition is formulated for oral, subcutaneous (s.c.), intradermal, intramuscular, intraperitoneal and/or intravenous (i.v.) administration. In certain embodiments, a pharmaceutical composition contains formulation materials for modifying, maintaining, or preserving, for example, the pH, osmolarity, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates (e.g., phosphate buffered saline) or suitable organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counter ions (such as sodium); solvents (such as glycerin, propylene glycol or polyethylene glycol); diluents; excipients and/or pharmaceutical adjuvants. In particular, a pharmaceutical composition can comprise any suitable carrier, formulation, or ingredient, the like or combinations thereof as listed in “Remington: The Science And Practice Of Pharmacy” Mack Publishing Co., Easton, PA, 19th Edition, (1995) (hereafter, Remington '95), or “Remington: The Science And Practice Of Pharmacy”, Pharmaceutical Press, Easton, PA, 22^{nd} Edition, (2013) (hereafter, Remington 2013), the contents of which are incorporated herein by reference in their entirety.

In certain embodiments, a pharmaceutical composition comprises a suitable excipient, non-limiting examples of which include anti-adherents (e.g., magnesium stearate), a binder, fillers, monosaccharides, disaccharides, other carbohydrates (e.g., glucose, mannose or dextrin), sugar alcohols (e.g., mannitol or sorbitol), coatings (e.g., cellulose, hydroxypropyl methylcellulose (HPMC), microcrystalline cellulose, synthetic polymers, shellac, gelatin, corn protein zein, enterics or other polysaccharides), starch (e.g., potato, maize or wheat starch), silica, colors, disintegrants, flavors, lubricants, preservatives, sorbents, sweeteners, vehicles, suspending agents, surfactants and/or wetting agents (such as pluronics, PEG, sorbitan esters, polysorbates such as polysorbate 20, polysorbate 80, triton, tromethamine, lecithin, cholesterol, tyloxapal), stability enhancing agents (such as sucrose or sorbitol), and tonicity enhancing agents (such as alkali metal halides, sodium or potassium chloride, mannitol, sorbitol), and/or any excipient disclosed in Remington '95 or Remington 2013. The term “binder” as used herein refers to a compound or ingredient that helps keeps a pharmaceutical mixture combined. Suitable binders for making pharmaceutical formulations and are often used in the preparation of pharmaceutical tablets, capsules and granules are known to those skilled in the art.

In some embodiments a pharmaceutical composition comprises a suitable pharmaceutically acceptable additive and/or carrier. Non-limiting examples of suitable additives include a suitable pH adjuster, a soothing agent, a buffer, a sulfur-containing reducing agent, an antioxidant and the like. Non-limiting examples of a sulfur-containing reducing agent include those having a sulfhydryl group (e.g., a thiol) such as N-acetylcysteine, N-acetylhomocysteine, thioctic acid, thiodiglycol, thioethanolamine, thioglycerol, thiosorbitol, thioglycolic acid and a salt thereof, sodium thiosulfate, glutathione, and a C1-C7 thioalkanoic acid. Non-limiting examples of an antioxidant include erythorbic acid, dibutylhydroxytoluene, butylhydroxyanisole, alpha-tocopherol, tocopherol acetate, L-ascorbic acid and a salt thereof, L-ascorbyl palmitate, L-ascorbyl stearate, sodium bisulfite, sodium sulfite, triamyl gallate and propyl gallate, as well as chelating agents such as disodium ethylenediaminetetraacetate (EDTA), sodium pyrophosphate and sodium metaphosphate. Furthermore, diluents, additives and excipients may comprise other commonly used ingredients, for example, inorganic salts such as sodium chloride, potassium chloride, calcium chloride, sodium phosphate, potassium phosphate and sodium bicarbonate, as well as organic salts such as sodium citrate, potassium citrate and sodium acetate.

The pharmaceutical compositions used herein can be stable over an extended period of time, for example on the order of months or years. In some embodiments a pharmaceutical composition comprises one or more suitable preservatives. Non-limiting examples of preservatives include benzalkonium chloride, benzoic acid, salicylic acid, thimerosal, phenethyl alcohol, methylparaben, propylparaben, chlorhexidine, sorbic acid, hydrogen peroxide, the like and/or combinations thereof. A preservative can comprise a quaternary ammonium compound, such as benzalkonium chloride, benzoxonium chloride, benzethonium chloride, cetrimide, sepazonium chloride, cetylpyridinium chloride, or domiphen bromide (BRADOSOL®). A preservative can comprise an alkyl-mercury salt of thiosalicylic acid, such as thimerosal, phenylmercuric nitrate, phenylmercuric acetate or phenylmercuric borate. A preservative can comprise a paraben, such as methylparaben or propylparaben. A preservative can comprise an alcohol, such as chlorobutanol, benzyl alcohol or phenyl ethyl alcohol. A preservative can comprise a biguanide derivative, such as chlorohexidine or polyhexamethylene biguanide. A preservative can comprise sodium perborate, imidazolidinyl urea, and/or sorbic acid. A preservative can comprise stabilized oxychloro complexes, such as known and commercially available under the trade name PURITE®. A preservative can comprise polyglycol-polyamine condensation resins, such as known and commercially available under the trade name POLYQUART® from Henkel KGaA. A preservative can comprise stabilized hydrogen peroxide. A preservative can be benzalkonium chloride. In some embodiments a pharmaceutical composition is free of preservatives.

In some embodiments a composition, pharmaceutical composition or compound for use in a method described herein is substantially free of contaminants (e.g., blood cells, platelets, polypeptides, minerals, blood-borne compounds or chemicals, virus, bacteria, other pathogens, toxin, and the like). In some embodiments a composition, pharmaceutical composition or compound for use in a method described herein is substantially free of serum and serum contaminants (e.g., serum proteins, serum lipids, serum carbohydrates, serum antigens and the like). In some embodiments a composition, pharmaceutical composition or compound for use in a method described herein is substantially free of a pathogen (e.g., a virus, parasite or bacteria). In some embodiments a composition, pharmaceutical composition or compound for use in a method described herein is substantially free of endotoxin. In some embodiments a composition, pharmaceutical composition or compound for use in a method described herein is substantially sterile. In some embodiments a composition, pharmaceutical composition or compound for use in a method described herein is sterile. In certain embodiments, a composition or pharmaceutical composition disclosed herein comprises a compound of Formula I, II, III or IV.

The pharmaceutical compositions described herein may be configured for administration to a subject in any suitable form and/or amount according to the use in which they are employed. For example, a pharmaceutical composition configured for parenteral administration (e.g., by injection or infusion), may take the form of a suspension, solution or emulsion in an oily or aqueous vehicle and it may contain formulation agents, excipients, additives and/or diluents such as aqueous or non-aqueous solvents, co-solvents, suspending solutions, preservatives, stabilizing agents and or dispersing agents. In some embodiments a pharmaceutical composition suitable for parenteral administration may contain one or more excipients. In some embodiments a pharmaceutical composition is lyophilized to a dry powder form. In some embodiments a pharmaceutical composition is lyophilized to a dry powder form, which is suitable for reconstitution with a suitable pharmaceutical solvent (e.g., water, saline, an isotonic buffer solution (e.g., PBS), DMSO, combinations thereof and the like). In certain embodiments, reconstituted forms of a lyophilized pharmaceutical composition are suitable for parenteral administration (e.g., intravenous administration) to a mammal.

In certain embodiments, a pharmaceutical composition is configured for oral administration and may be formulated as a tablet, microtablet, minitablets, micropellets, powder, granules, capsules (e.g., capsules filled with microtablets, micropellets, powders or granules), emulsions, solutions, the like or combinations thereof. Pharmaceutical compositions configured for oral administration may comprise suitable coatings to delay or sustain release of the active ingredient, non-limiting examples of which include enteric coatings such as fatty acids, waxes, shellac, plastics, methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, cellulose acetate trimellitate, sodium alginate, zein, plant fibers, the like and combinations thereof.

In some embodiments a pharmaceutical compositions described herein may be configured for topical administration and may include one or more of a binding and/or lubricating agent, polymeric glycols, gelatins, cocoa-butter or other suitable waxes or fats. In some embodiments a pharmaceutical composition described herein is incorporated into a topical formulation containing a topical carrier that is generally suited to topical drug administration and comprising any suitable material known to those skilled in the art. In certain embodiments, a topical formulation of a pharmaceutical composition is formulated for administration of a compound for use in a method described herein from a topical patch.

In some embodiments a pharmaceutical compositions described herein may be configured for contacting an organ and/or for perfusion of an organ. For example, in some embodiment, a pharmaceutical composition configured for contacting an organ is substantially sterile, comprises one or more compounds disclosed herein, and comprises a solution of salts that is isotonic with the body fluids of a subject.

In certain embodiments, an optimal pharmaceutical composition is determined by one skilled in the art depending upon, for example, on the intended route of administration, delivery format and desired dosage (see e.g., Remington '95 or Remington 2013, supra). A pharmaceutical composition can be manufactured by any suitable manner, including, e.g., by means of conventional mixing, dissolving, granulating, dragee-making, levigating, emulsifying, encapsulating, entrapping or tableting processes (e.g., see methods described in Remington '95 or Remington 2013).

In some embodiments, a composition further comprises an immunosuppressive agent, non-limiting examples of which include a calcineurin inhibitor, a cyclosporin (e.g., cyclosporin A), an antiproliferative agent, a steroid (e.g., a corticosteroid), cyclophilin D, a RIPK1 kinase inhibitor (e.g., Necrostatin-1 (Nec-1)), an immunoglobulin G degrading enzyme (e.g., Imlifidase (IdeS)), heme arginate, an anti-inflammatory agent, combinations thereof and the like.

### Route of Administration

Any suitable method of administering a composition, pharmaceutical composition or compound for use in a method described herein to a subject can be used. Any suitable formulation and/or route of administration can be used for administration of a compound for use in a method described herein or composition disclosed herein (e.g., see Fingl et al. 1975, in “The Pharmacological Basis of Therapeutics”, which is incorporated herein by reference in its entirety). A suitable formulation and/or route of administration can be chosen by a medical professional (e.g., a physician) in view of, for example, a subject's risk, age, and/or condition. Non-limiting examples of routes of administration include topical or local (e.g., transdermally or cutaneously, (e.g., on the skin or epidermis), in or on the eye, intranasally, transmucosally, in the ear, inside the ear (e.g., behind the ear drum)), enteral (e.g., delivered through the gastrointestinal tract, e.g., orally (e.g., as a tablet, capsule, granule, liquid, emulsification, lozenge, or combination thereof), sublingual, by gastric feeding tube, rectally, and the like), by parenteral administration (e.g., parenterally, e.g., intravenously, intra-arterially, intramuscularly, intraperitoneally, intradermally, subcutaneously, intracavity, intracranial, intra-articular, into a joint space, intracardiac (into the heart), intracavernous injection, intralesional (into a skin lesion), intraosseous infusion (into the bone marrow), intrathecal (into the spinal canal), intrauterine, intravaginal, intravesical infusion, intravitreal), the like or combinations thereof.

In some embodiments a compound for use in a method described herein or pharmaceutical composition described herein is administered to the lungs, bronchial passages, trachea, esophagus, sinuses, or nasal passages using a suitable method, non-limiting examples of which include intranasal administration, intratracheal instillation, and oral inhalative administration (e.g., by use of an inhaler, e.g., single/-multiple dose dry powder inhalers, nebulizers, and the like).

In some embodiments a compound for use in a method described herein or a pharmaceutical composition disclosed herein is provided to a subject. For example, a composition that is provided to a subject is sometimes provided to a subject for self-administration or for administration to a subject by another (e.g., a non-medical professional). As another example, a composition can be provided as an instruction written by a medical practitioner that authorizes a patient to be provided a composition or treatment described herein (e.g., a prescription). In yet another example, a composition can be provided to a subject where the subject self-administers a composition orally, intravenously or by way of an inhaler, for example.

Alternately, one can administer a compound for use in a method described herein or composition in a local rather than systemic manner, for example, via direct application to the skin, mucous membrane or region of interest for treating, including using a depot or sustained release formulation.

In certain embodiments a pharmaceutical composition comprising a compound for use in a method described herein is administered alone (e.g., as a single active ingredient (AI or e.g., as a single active pharmaceutical ingredient (API)). In other embodiments, a pharmaceutical composition comprising a compound for use in a method described herein is administered in combination with one or more additional Als/APIs, for example, as two separate compositions or as a single composition where the one or more additional AIs/APIs are mixed or formulated together with a compound for use in a method described herein in a pharmaceutical composition.

### Dose and Effective Amounts

In some embodiments, an amount of a compound for use in a method described herein (e.g., in a pharmaceutical composition) is a therapeutically effective amount. In certain embodiments, a pharmaceutical composition comprises a therapeutically effective amount of a compound disclosed herein. In some embodiments, a therapeutically effective amount of a compound for use in a method described herein is administered to a subject. In some embodiments, a therapeutically effective amount of a compound for use in a method described herein is an amount needed to obtain an effective therapeutic outcome. In certain embodiments, a therapeutically effective amount of a compound for use in a method described herein is an amount sufficient to inhibit, delay, reduce or prevent rejection of a transplanted organ. In certain embodiments, an effective amount of a compound for use in a method described herein is an amount sufficient to inhibit, delay, reduce or prevent injury (e.g., reperfusion injury), necrosis or cell death of an organ as contemplated herein. Determination of an effective amount or a therapeutically effective amount is well within the capability of those skilled in the art, especially in light of the detailed disclosure provided herein.

In certain embodiments, a therapeutically effective amount is an amount high enough to provide an effective therapeutic effect (e.g., a beneficial therapeutic effect) and an amount low enough to minimize unwanted adverse reactions. Accordingly, in certain embodiments, a therapeutically effective amount of a compound for use in a method described herein may vary from subject to subject, often depending on age, weight, general health condition of a subject, amount of fluid loss due to trauma or surgery, and/or a particular combination of drugs administered to a subject. Thus, in some embodiments, a therapeutically effective amount is determined empirically. Accordingly, a therapeutically effective amount of a compound for use in a method described herein that is administered to a subject can be determined by one of ordinary skill in the art based on amounts found effective in animal or clinical studies, a physician's experience, and suggested dose ranges or dosing guidelines, for example.

In certain embodiments, a therapeutically effective amount of a compound for use in a method described herein is administered at a suitable dose (e.g., at a suitable volume, frequency and/or concentration, which often depends on a subject's weight, age and/or condition) intended to obtain an acceptable therapeutic outcome. In certain embodiments, a therapeutically effective amount of a compound for use in a method described herein comprises one or more doses selected from at least 0.01 mg/kg (e.g., mg of a compound for use in a method described herein per kg body weight of a subject), at least 0.1 mg/kg, at least 0.5 mg/kg, at least 1 mg/kg, at least 10 mg/kg or at least 100 mg/kg. In certain embodiments, a therapeutically effective amount of a compound for use in a method described herein is selected from one or more doses of about 0.001 mg/kg (e.g., mg of a compound for use in a method described herein per kg body weight of a subject) to about 5000 mg/kg, 0.01 mg/kg to 1000 mg/kg, 0.01 mg/kg to 500 mg/kg, 0.1 mg/kg to 1000 mg/kg, 1 mg/kg to 1000 mg/kg, 10 mg/kg to 1000 mg/kg, 100 mg/kg to 1000 mg/kg, 0.1 mg/kg to 500 mg/kg, 0.1 mg/kg to 250 mg/kg, 0.1 mg/kg to 150 mg/kg, 0.1 mg/kg to 100 mg/kg, 0.1 mg/kg to 75 mg/kg, 0.1 mg/kg to 50 mg/kg, 0.1 mg/kg to 25 mg/kg, 0.1 mg/kg to 10 mg/kg, 0.1 mg/kg to 5 mg/kg, 0.5 mg/kg to 5 mg/kg, intervening amounts and combinations thereof. In some aspects a therapeutically effective amount of a compound for use in a method described herein administered to a subject comprises one or more doses of about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 50 mg/kg, 100 mg/kg, 500 mg/kg, and intervening amounts and combinations thereof. In some embodiments a therapeutically effective amount of a compound for use in a method described herein is between about 0.1 mg/kg and about 50 mg/kg.

In some embodiments administering a therapeutically effective amount of a compound for use in a method described herein, or a pharmaceutical composition comprising a compound for use in a method described herein, comprises administering a suitable dose at a frequency or interval as needed to obtain an effective therapeutic outcome. In some embodiments administering a therapeutically effective amount of a compound for use in a method described herein or a pharmaceutical composition disclosed herein comprises administering a suitable dose hourly, every two hours, every 4 hours, every 6 hours, three times a day, twice a day, once a day, six times a week, five times a week, four times a week, three times a week, twice a week, weekly, at combinations thereof, and/or at regular or irregular intervals thereof, and/or simply at a frequency or interval as needed or recommended by a medical professional. In some embodiments, a therapeutically effective amount of a compound for use in a method described herein or a pharmaceutical composition comprising a therapeutically effective amount of compound for use in a method described herein is administered continuously by, for example by intravenous administration.

In some embodiments a therapeutically effective amount of a compound for use in a method described herein is administered to a subject prior to, perioperatively, during and/or after an organ transplant procedure. In some embodiments a therapeutically effective amount of a compound for use in a method described herein is administered to a subject up to 3 days prior to, up to 2 days prior to, up to 1 day prior to, up to 20 hours prior to, up to 15 hours prior to, up to 10 hours prior to, up to 5 hours prior to, up to 2 hours prior to or up to 1 hour prior to an organ transplant procedure. In some embodiments a therapeutically effective amount of a compound for use in a method described herein is administered to a subject 0 to 72 hours, 0 and 48 hours, 0 to 24 hours, 0 to 12 hours, 0 to 6 hours, 0 to 4 hours, or 0 to 2 hours before an organ transplant procedure. In some embodiments a therapeutically effective amount of a compound for use in a method described herein is administered perioperatively. In some embodiments a therapeutically effective amount of a compound for use in a method described herein is during an organ transplant procedure. In some embodiments a therapeutically effective amount of a compound for use in a method described herein is administered intermittently or continuously for up to 1 hour after, 2 hours after, 4 hours after, 6 hours after, 12 hours after, 24 hours after, 2 days after, 3 days after, a week after, 1 month after, 3 months after, 6 months after, 12 months after, 18 months after, 24 months after or up to 36 months after an organ transplant procedure. In some embodiments a therapeutically effective amount of a compound for use in a method described herein is administered for several years after an organ transplant procedure and/or until death of a subject.

In certain embodiments, an effective amount of a compound for use in a method described herein is selected from a concentration in a range of 0.001 μg/ml to 2 g/ml, 0.001 μg/ml to 1000 mg/ml, 0.001 μg/ml to 500 mg/ml, 0.001 μg/ml to 100 mg/ml, 0.001 μg/ml to 50 mg/ml, 0.01 μg/ml to 50 mg/ml, 0.1 μg/ml to 50 mg/ml, 1 μg/ml to 50 mg/ml, 50 μg/ml to 50 mg/ml, and intermediate concentrations therein.

### Kits

In some embodiments, provided herein is a kit comprising a compound for use in a method described herein or a pharmaceutical composition comprising a compound for use in a method described herein. In some embodiments, a kit comprises one or more containers comprising a composition disclosed herein, where each container contains an amount or dose of a compound for use in a method described herein. In some embodiments, a kit comprises one or more packs and/or one or more dispensing devices, which can contain one or more amounts or doses of a compound or composition for use in a method described herein. Non-limiting examples of a pack include a metal, glass, or plastic container, syringe or blister pack that comprises a compound for use in a method described herein or a composition described herein. In certain embodiments, a kit comprises a dispensing device such as a syringe or inhaler, that may or may not comprise a compound for use in a method described herein or a composition described herein.

In some embodiments, a kit comprises a flexible container or i.v. bag comprising a sterile isotonic pharmaceutical composition suitable for intravenous administration comprising a compound described herein at a concentration in a range of 0.001 μg/ml to 100 mg/ml.

In some embodiments a kit, container or pack comprises an amount of a compound for a single use or, for example, a single perfusion. In some embodiments a kit, container or pack comprises an amount of a compound for use in a method described herein sufficient to administer to a subject for 1 day to 1 year, 1 day to 180 days, 1 day to 120 days, 1 day to 90 days, 1 day to 60 days, 1 day to 30 days, 1-24 hours, 1-12 hours, 1-4 hours, or an amount of time there between.

A kit optionally includes a product label and/or one or more packaging inserts, that provide a description of the components or instructions for use in vitro, in vivo, ex vivo or in situ of the components therein. Exemplary instructions may include instructions for administration protocol or therapeutic regimen. In certain embodiments, a kit comprises packaging material, which refers to a physical structure housing components of the kit. The packaging material can maintain the components sterilely and can be made of material commonly used for such purposes (e.g., paper, corrugated fiber, glass, plastic, foil, ampules, vials, tubes, etc.). Product labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, optical disk such as CD- or DVD-ROM/RAM, DVD, MP3, magnetic tape, or an electrical storage media such as RAM and ROM or hybrids of these such as magnetic/optical storage media, FLASH media or memory-type cards. Product labels or inserts can include identifying information of one or more components therein, dose amounts, clinical pharmacology of the active ingredient(s) including mechanism of action, pharmacokinetics (PK) and pharmacodynamics (PD). Product labels or inserts can include information identifying manufacturer information, lot numbers, manufacturer location, date, information on an indicated condition, disorder, disease or symptom for which a kit component may be used. Product labels or inserts can include instructions for the clinician or for a subject for using one or more of the kit components in a method, treatment protocol or therapeutic regimen. Instructions can include dosage amounts, frequency or duration, and instructions for practicing any of the methods set forth herein. Product labels or inserts can include information on potential adverse side effects and/or warnings. A pack and/or dispenser device can be accompanied by instructions for administration. The pack or dispenser can also be accompanied with a notice associated with the container in a form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert.

### EXAMPLES

### Example 1—Assay for Necroptosis & Apoptosis

In this Example, the therapeutic potential of a compound described herein for inhibiting necroptosis or apoptosis is determined by a method described in Gan et al. (2018) Am. J. Transplant. 19: 686-698. Briefly, B6 and Cyp-D^{-/-} microvascular cells (MVECs) are grown in complete endothelial growth media-2 (EGM-2 medium) supplemented with fetal bovine serum and growth factors in 96-well flat-bottomed plates for 24 hours to confluency. Test and control reagents are added to each well and cell death is assessed as a measure of either SYTOX Green Nucleic Acid Stain (100 nmol/L; Thermo Fisher Scientific, Waltham, MA) or 7-aminoactinomycin D (eBioscience). Cell death is monitored using the IncuCyte Zoom System (Essen BioScience, Ann Arbor, MI) or the CytoFLEX flow cytometer (Beckman Coulter, Brea, CA).

Cells are treated with 100 ng/ml tumor necrosis factor and smac-mimetic (100 nmol/L) to induce cell death, with or without a caspase-8 inhibitor (i.e., Z-Ile-Glu-Asp-fluoromethylketoe (10-30 μmol/L)), a caspase-3 inhibitor (Z-Asp-Glu-Val-Asp-fluoromethylketoe (DEVD, 10-30 μmol/L)), and/or a caspase-9 inhibitor (Z-Leu-Glu-His-Asp-fluoromethylketoe (LEHD, 10-30 μmol/L)) to inhibit caspase-mediated apoptosis. The smac-mimetic reagent is added to suppress the function of inhibitor of apoptosis proteins (IAP) which promotes apoptosis. Cells are treated with a RIPK1 inhibitor (necrostatin-1s (NEC-1s, 10-30 μmol/L)) and/or an MLKL inhibitor (GW806742X (50-1000 nmol/L, SYNkinase, San Diego, CA)) to inhibit necroptosis. Cells are treated with S-15176 (Sigma-Aldrich) to inhibit mitochondrial permeability transition events. Cyclosporin A (CsA) is added as a positive control as an inhibitor of TNF-induced necroptotic cell death. A compound of Formula I, II, III or IV are added as test reagents to assay their potential to inhibit necroptosis and/or apoptosis.

### Example 2—Certain Non-Limiting Embodiments

A1. A method for inhibiting, reducing, delaying or preventing rejection of a transplanted organ comprising administering to a subject a composition comprising a therapeutically effective amount of a compound having the structure of Formula I;

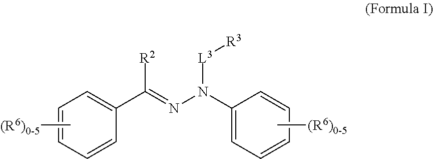

or a pharmaceutically acceptable salt, stereoisomer or tautomer thereof,
wherein:
   R² is selected from the group consisting of H and methyl;
   R³ is trifluoromethyl or other fluoro substituted alkyl;
   L³ is a carbonyl; and
   R⁶ at each occurrence is independently selected from the group consisting of alkyl, substituted alkyl, cycloalkyl, substituted cycloalkyl, hydroxyl, alkoxy, substituted alkoxy, aryloxy, substituted aryloxy, mercapto, alkylthio, arylthio, carbonyl, aryl, substituted aryl, substituted heterocyclic, halogen, cyano, cyanoalkyl, nitro, amino, amidino, carbamate, S(O)ₙR⁷ and C(O)R⁸ or two R⁶ at adjacent positions combine to form an optionally substituted heteroaryl or heteroalkyl ring fused with the adjoining phenyl moiety;
   R⁷ is H, R⁹, NH₂, HNR⁹ or NR⁹R¹⁰;
   R⁸ is OH, OR⁹, NH₂, NHR⁹ or NR⁹R¹⁰;
   R⁹ and R¹⁰ at each occurrence are independently optionally substituted alkyl; and
   n=1 or 2.
A2. The method of embodiment A1, wherein R⁶ at each occurrence is selected from the group consisting of alkyl, substituted alkyl, hydroxyl, alkoxy, substituted alkoxy, halogen, and C(O)R⁸.
A3. The method of embodiment A 2, wherein R⁶ at each occurrence is selected from the group consisting of methyl, methoxy, perfluoromethyl, perfluoromethoxy, hydroxyl, Cl, F, and I.
A4. The method of embodiment A1, wherein the compound has the structure of Formula II;

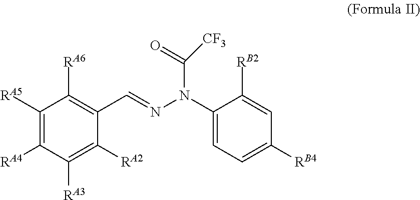

or a pharmaceutically acceptable salt, stereoisomer or tautomer thereof, wherein:
(i) R^{A2}, R^{A4}, R^{A5}, and R^{A6} is H, R^{A3} is methoxy, R^{B2} is methyl, and R^{B4} is methyl; or
(ii) R^{A2}, R^{A3}, R^{A5}, and R^{A6} is H, R^{A4} is methoxy, R^{B2} is methyl, and R^{B4} is methyl; or
(iii) R^{A2}, R^{A3}, R^{A4}, R^{A5}, and R^{A6} is H, R^{B2} is H, and R^{B4} is H; or
(iv) R^{A2}, R^{A3}, R^{A4}, R^{A5}, and R^{A6} is H, R^{B2} is methyl, and R^{B4} is methyl; or
(v) R^{A2}, R^{A4}, R^{A5}, and R^{A6} is H, R^{A3} is methoxy, R^{B2} is H, and R^{B4} is H; or
(vi) R^{A2}, R^{A3}, R^{A4}, R^{A5}, and R^{A6} is H, R^{B2} is H, and R^{B4} is methyl; or
(vii) RAZ, R^{A4}, R^{A5}, and R^{A6} is H, R^{A3} is methoxy, R^{B2} is H, and R^{B4} is methyl; or
(viii) R^{A2}, R^{A3}, R^{A4}, R^{A5}, and R^{A6} is H, R^{B2} is methyl, and R^{B4} is H; or
(ix) R^{A2}, R^{A4}, R^{A5}, and R^{A6} is H, R^{A3} is methoxy, R^{B2} is methyl, and R^{B4} is H; or
(x) R^{A2}, R^{A3}, R^{A5}, and R^{A6} is H, R^{A4} is COOH, R^{B2} is methyl, and R^{B4} is methyl; or
(xi) R^{A2}, R^{A4}, and R^{A5} is H, R^{A3} and R^{A6} is hydroxyl, R^{B2} is methyl, and R^{B4} is methyl; or
(xii) R^{A2}, R^{A4}, and R^{A6} is H, R^{A3} and R^{A5} is hydroxyl, R^{B2} is methyl, and R^{B4} is methyl; or
(xiii) R^{A2}, R^{A4}, and R^{A5} is H, R^{A3} is methoxy, R^{A6} is F, R^{B2} is H, and R^{B4} is Cl; or (xiv) R^{A3} and R^{A5} is H, R^{A2} and R^{A6} is F, R^{A4} is hydroxyl, R^{A6} is F, R^{B2} is H, and R^{B4} is F; or
(xv) R^{A2}, R^{A4}, and R^{A6} is H, R^{A3} is hydroxyl, R^{A5} is F, R^{B2} is H, and R^{B4} is F; or
(xvi) R^{A2}, R^{A5}, and R^{A6} is H, R^{A3} and R^{A4} taken together are —O—CH₂—O—, R^{A5} is F, R^{B2} is H, and R^{B4} is F.
A5. The method of embodiment A4, wherein R^{A2}, R^{A4}, R^{A5}, and R^{A6} is H, R^{A3} is methoxy, R^{B2} is methyl, and R^{B4} is methyl.
A6. The method of any one of embodiments A1 to A5, wherein the subject is an organ transplant recipient.
A7. The method of any one of embodiments A1 to A5, wherein the subject is an organ donor.
B1. A method of inhibiting, reducing, delaying or preventing rejection of a transplanted organ comprising administering to a subject a composition comprising a therapeutically effective amount of a compound comprising a structure selected from any one of Formulas I to IV, or a pharmaceutically acceptable salt, stereoisomer or tautomer thereof.
B2. The method of embodiment B1, wherein the composition comprises a therapeutically effective amount of the compound comprising the structure of Formula IV;

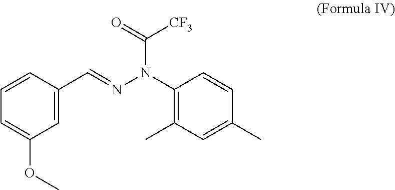 [SMILES: COc1cccc(/C=N/N(C(=O)C(F)(F)F)c2ccc(C)cc2C)c1]

or a pharmaceutically acceptable salt, stereoisomer or tautomer thereof.
B3. The method of embodiment B1 or B2, wherein the subject is an organ transplant recipient.
B4. The method of embodiment B1, B2 or B3, wherein the subject is in need of an organ transplant.
B5. The method of any one of embodiments B1 to B4, wherein the composition is administered during, prior to, or after transplant of the organ into the subject.
B5.1. The method of embodiment B1 or B2, wherein the subject is an organ donor.
B5.2. The method of embodiments B5.1, wherein the composition is administered prior to transplant of the organ into an organ transplant recipient.
B6. The method of any one of embodiments B1 to B5.2, wherein the rejection that is inhibited, reduced, delayed or prevented is acute transplant rejection.
B7. The method of any one of embodiments B1 to B5, wherein the rejection that is inhibited, reduced, delayed or prevented is chronic transplant rejection.
C1. A ex vivo or in vitro method of preparing an organ for transplantation into an organ transplant recipient comprising contacting the organ with a composition comprising an effective amount of a compound comprising a structure selected from any one of Formulas I to IV, or a pharmaceutically acceptable salt, stereoisomer or tautomer thereof.
C2. The method of embodiment C1, wherein the organ in not attached or connected to a subject.
C3. The method of embodiment C1 or C2, wherein the organ is separated or removed from a subject.
C4. The method of any one of embodiments C1 to C3, wherein the contacting comprises perfusion of the organ with the composition.
D1. An in situ method of preparing an organ for transplantation comprising contacting the organ of an organ donor with a composition comprising an effective amount of a compound comprising a structure selected from any one of Formulas I to IV, or a pharmaceutically acceptable salt, stereoisomer or tautomer thereof.
D2. The method of embodiment D1, wherein the organ is contacted with the composition prior to removal from the organ donor.
D3. The method of embodiment D1 or D2, wherein the organ is operably connected to the organ donor.
D4. The method of any one of embodiments D1 to D3, wherein the contacting comprises perfusion of the organ with the composition.
D5. The method of any one of embodiments D1 to D4, wherein the organ donor is alive.
D6. The method of any one of embodiments D1 to D5, wherein the organ donor is brain dead.
D7. The method of any one of embodiments D1 to D5, wherein the organ donor is deceased.
E1. A method of inhibiting, reducing, delaying or preventing reperfusion injury of a transplanted organ in a subject comprising administering to the subject a composition comprising a therapeutically effective amount of a compound comprising a structure selected from any one of Formulas I to IV, or a pharmaceutically acceptable salt, stereoisomer or tautomer thereof.
E2. The method of embodiments E1, wherein the composition is administered the subject during, prior to, or after transplant of the organ into the subject.
E3. The method of E1 or E2, wherein the subject is an organ transplant recipient.
F1. A method of inhibiting, reducing, delaying or preventing cell death of cells in an organ comprising administering to the organ or to a subject operably connected to the organ, a composition comprising a therapeutically effective amount of a compound comprising a structure selected from any one of Formulas I to IV, or a pharmaceutically acceptable salt, stereoisomer or tautomer thereof.
F2. The method of embodiments F1, wherein the composition is administered during, prior to, or after transplant of the organ into an organ transplant recipient.
F2.1. The method of embodiment F1 or F2, wherein the subject is an organ donor.
F2.2. The method of embodiment F1 or F2, wherein the subject is an organ transplant recipient.
F3. The method of embodiment F1 to F2.2, wherein the cell death comprises apoptosis, necrosis, necroptosis or autophagy.
G1. A ex vivo or in vitro method of inhibiting, reducing, delaying or preventing cell death of cells in an organ comprising contacting the organ with a composition comprising an effective amount of a compound comprising a structure selected from any one of Formulas I to IV, or a pharmaceutically acceptable salt, stereoisomer or tautomer thereof.
G2. The method of embodiment G1, wherein the organ in not attached or connected to a subject.
G2.2. The method of embodiment G1 or G2, wherein the method is conducted after removal of the organ from an organ donor and prior to transplantation of the organ into an organ transplant recipient.
G3. The method of any one of embodiments G1 to G2.2, wherein the contacting comprises perfusion of the organ with the composition.
G4. The method of any one of embodiments G1 to G3, wherein the cell death comprises apoptosis, necrosis, necroptosis or autophagy.
H1. An in situ method of inhibiting, reducing, delaying or preventing cell death of cells in an organ comprising contacting the organ of a subject with a composition comprising an effective amount of a compound comprising a structure selected from any one of Formulas I to IV.
H2. The method of embodiment H1, wherein the subject is an organ donor and the organ is contacted with the composition prior to removal of the organ from the organ donor.
H3. The method of embodiment H1 or H2, wherein the organ is operably connected to the subject or to the organ donor.
H4. The method of any one of embodiments H1 to H3, wherein the contacting comprises perfusion of the organ with the composition.
H5. The method of any one of embodiments H1 to H4, wherein the subject or organ donor is alive.
H6. The method of any one of embodiments H1 to H5, wherein the subject or organ donor is brain dead.
H7. The method of any one of embodiments H1 to H6, wherein the cell death comprises apoptosis, necrosis, necroptosis or autophagy.
I1. A pharmaceutical composition comprising a compound comprising a structure selected from any one of Formula I, Formula II, Formula III and Formula IV, or a pharmaceutically acceptable salt, stereoisomer or tautomer thereof, for use in conducting a method of any one of embodiments A1 to H7.
J1. The method of any one of embodiments A1 to I1, wherein the organ is a solid organ, tissue, body part, or portion thereof.
J1.1. The method of embodiments J1, wherein the organ is a solid organ, or portion thereof.
J1.2. The method of embodiment J1.1, wherein the solid organ is selected from a kidney, liver, heart, lung, pancreas, intestine, or portion thereof.
J2. The method of any one of embodiments A1 to J1.2, wherein the subject is a mammal.
J3. The method of any one of embodiments A1 to J1.2, wherein the subject is a primate.
J4. The method of any one of embodiments A1 to J1.2, wherein the subject is a human.
J5. The method of any one of embodiments A1 to J4, wherein the composition further comprises an immunosuppressive agent.
J6. The method of embodiment J5, wherein the immunosuppressive agent is selected from one or more of a calcineurin inhibitor, a cyclosporin, cyclosporin A, an antiproliferative agent, a steroid, a corticosteroid, cyclophilin D, Necrostatin-1 (Nec-1), Imlifidase, heme arginate, and an anti-inflammatory agent.
K1 The method for inhibiting, reducing, delaying or preventing rejection, injury or cell death of a transplanted organ in a subject comprising administering to the subject a composition comprising a therapeutically effective amount of a compound having the structure of Formula IV or a pharmaceutically acceptable salt, stereoisomer or tautomer thereof.
K2. The ex vivo or in vitro method of preparing an organ for transplantation into a subject comprising contacting the organ with a composition comprising an effective amount of a compound that comprises the structure of Formula IV or a pharmaceutically acceptable salt, stereoisomer or tautomer thereof.
K3. The in situ or in vivo method of preparing an organ for transplantation comprising contacting the organ prior to removal from a donor subject with a composition comprising an effective amount of a compound that comprises the structure of Formula IV or a pharmaceutically acceptable salt, stereoisomer or tautomer thereof.
K4. The method of inhibiting, reducing, delaying or preventing reperfusion injury of a transplanted organ in a subject comprising administering to the subject a composition comprising a therapeutically effective amount of a compound that comprises the structure of Formula IV or a pharmaceutically acceptable salt, stereoisomer or tautomer thereof.
K5. The method of inhibiting, reducing, delaying or preventing cell death of cells in a transplanted organ in a subject comprising administering to the subject a composition comprising a therapeutically effective amount of a compound that comprises the structure of Formula IV or a pharmaceutically acceptable salt, stereoisomer or tautomer thereof.
K6. The ex vivo or in vitro method of inhibiting, reducing, delaying or preventing cell death of cells in an organ comprising contacting the organ with a composition comprising an effective amount of a compound that comprises the structure of Formula IV or a pharmaceutically acceptable salt, stereoisomer or tautomer thereof.
K7. The in situ method of inhibiting, reducing, delaying or preventing cell death of cells in an organ comprising contacting the organ prior to removal from a donor subject with a composition comprising an effective amount of a compound that comprises the structure of Formula IV or a pharmaceutically acceptable salt, stereoisomer or tautomer thereof.
K8. The method of embodiment K1 wherein an effective amount of the compound is administered to the organ prior to its transplantation into the subject.
K9. The method of embodiment K8 wherein the compound having the structure of Formula IV is administered to the organ prior its removal from the donor subject.
K10. The method of embodiment K8 or K9 wherein the compound having the structure of Formula IV is administered to the organ after its removal from the donor subject and prior to its transplantation into the subject.
K11. The method of embodiment K1, K3, K4, K5, K7, K8-K10 wherein the compound is administered to the subject intravenously or intra-arterially.
K12. The method of embodiment K11 wherein the compound is additionally administered to the subject orally prior to and/or after receipt of the transplanted organ.
K13. The method of embodiment K2, K6, K8-K10 wherein the compound is administered to the organ locally and/or in a perfusion fluid.

The entirety of each patent, patent application, publication or any other reference or document cited herein hereby is incorporated by reference. In case of conflict, the specification, including definitions, will control.

Citation of any patent, patent application, publication or any other document is not an admission that any of the foregoing is pertinent prior art, nor does it constitute any admission as to the contents or date of these publications or documents.

Unless otherwise defined, all technical and scientific terms used herein have the same meaning as commonly understood by one of ordinary skill in the art.

All of the features disclosed herein may be combined in any combination. Each feature disclosed in the specification may be replaced by an alternative feature serving a same, equivalent, or similar purpose. Thus, unless expressly stated otherwise, disclosed features (e.g., antibodies) are an example of a genus of equivalent or similar features.

As used herein, all numerical values or numerical ranges include integers within such ranges and fractions of the values or the integers within ranges unless the context clearly indicates otherwise. Further, when a listing of values is described herein (e.g., about 50%, 60%, 70%, 80%, 85% or 86%) the listing includes all intermediate and fractional values thereof (e.g., 54%, 85.4%). Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth.

Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

Modifications can be made to the foregoing without departing from the basic aspects of the technology. Although the technology has been described in substantial detail with reference to one or more specific embodiments, those of ordinary skill in the art will recognize that changes can be made to the embodiments specifically disclosed in this application, yet these modifications and improvements are within the scope and spirit of the technology.

Some embodiments of the technology described herein suitably can be practiced in the absence of an element not specifically disclosed herein. Accordingly, in some embodiments the term “comprising” or “comprises” can be replaced with “consisting essentially of” or “consisting of” or grammatical variations thereof. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. The term, “substantially” as used herein refers to a value modifier meaning “at least 95%”, “at least 96%”, “at least 97%”, “at least 98%”, or “at least 99%” and may include 100%. For example, a composition that is substantially free of X, may include less than 5%, less than 4%, less than 3%, less than 2%, or less than 1% of X, and/or X may be absent or undetectable in the composition.

1. A method for inhibiting, reducing, delaying or preventing rejection, injury or cell death of a transplanted organ in a subject comprising administering to the subject a composition comprising a therapeutically effective amount of a compound having the structure of Formula I; 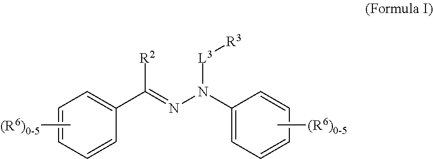
or a pharmaceutically acceptable salt, stereoisomer or tautomer thereof,
wherein:
R² is selected from the group consisting of H and methyl;
R³ is trifluoromethyl or other fluoro substituted alkyl;
L³ is a carbonyl; and
R⁶ at each occurrence is independently selected from the group consisting of alkyl, substituted alkyl, cycloalkyl, substituted cycloalkyl, hydroxyl, alkoxy, substituted alkoxy, aryloxy, substituted aryloxy, mercapto, alkylthio, arylthio, carbonyl, aryl, substituted aryl, substituted heterocyclic, halogen, cyano, cyanoalkyl, nitro, amino, amidino, carbamate, S(O)ₙR⁷ and C(O)R⁸ or two R⁶ at adjacent positions combine to form an optionally substituted heteroaryl or heteroalkyl ring fused with the adjoining phenyl moiety;
R⁷ is H, R⁹, NH₂, HNR⁹ or NR⁹R¹⁰;
R⁸ is OH, OR⁹, NH₂, NHR⁹ or NR⁹R¹⁰;
R⁹ and R¹⁰ at each occurrence are independently optionally substituted alkyl; and
n=1 or 2.

2. The method of claim 1, wherein the compound comprises the structure of Formula IV; 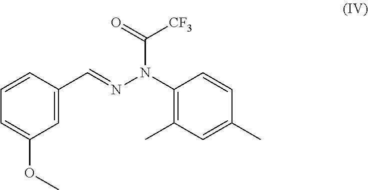 [SMILES: COc1cccc(/C=N/N(C(=O)C(F)(F)F)c2ccc(C)cc2C)c1]
or a pharmaceutically acceptable salt, stereoisomer or tautomer thereof.

3. A ex vivo or in vitro method of preparing an organ for transplantation into a subject comprising contacting the organ with a composition comprising an effective amount of a compound that comprises the structure of Formula IV; 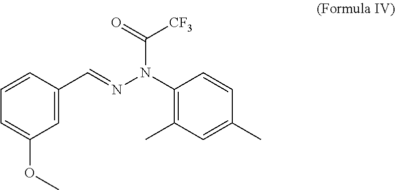 [SMILES: COc1cccc(/C=N/N(C(=O)C(F)(F)F)c2ccc(C)cc2C)c1]
or a pharmaceutically acceptable salt, stereoisomer or tautomer thereof.

4. An in situ or in vivo method of preparing an organ for transplantation comprising contacting the organ prior to removal from a donor subject with a composition comprising an effective amount of a compound that comprises the structure of Formula IV, 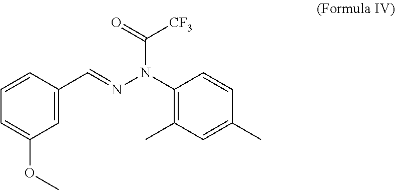 [SMILES: COc1cccc(/C=N/N(C(=O)C(F)(F)F)c2ccc(C)cc2C)c1]
or a pharmaceutically acceptable salt, stereoisomer or tautomer thereof.

5. A method of claim 2 wherein the administration of the compound to the subject inhibits, reduces, delays or prevents reperfusion injury of the transplanted organ in the subject.

6. The method of claim 2 wherein the administration of the compound to the subject inhibits, reduces, delays or prevents cell death of cells in the transplanted organ in the subject.

7. The method of claim 3 wherein contacting the organ with the compound inhibits, reduces, delays or prevents cell death of cells in the organ.

8. The method of claim 4 wherein the contacting the organ with the compound inhibits, reduces, delays or prevents cell death of cells in the organ.

9. The method of claim 2 wherein an effective amount of a compound having the structure of Formula IV is administered to the organ prior to its transplantation into the subject.

10. The method of claim 9 wherein the compound having the structure of Formula IV is administered to the organ prior its removal from the donor subject.

11. The method of claim 9 wherein the compound having the structure of Formula IV is administered to the organ after its removal from the donor subject and prior to its transplantation into the subject.

12. The method of claim 2, wherein the compound is administered to the subject intravenously or intra-arterially.

13. The method of claim 12 wherein the compound is additionally administered to the subject orally prior to and/or after receipt of the transplanted organ.

14. The method of claim 3, wherein the compound is administered to the organ locally and/or in a perfusion fluid.

15. The method of claim 9 wherein the compound is administered to the organ locally and/or in a perfusion fluid.

16. The method of claim 4 wherein the compound is administered to the donor subject intravenously or intra-arterially.
